(19) United States
(12) Patent Application Publication
(10) Pub. No.: US 2014/0088360 A1
(43) Pub. Date: Mar. 27, 2014

(54) MEDICAL DEVICE

(71) Applicant: TERUMO KABUSHIKI KAISHA, Shibuya-ku (JP)

(72) Inventor: Masakatsu KAWAURA, Hatanoshi (JP)

(73) Assignee: TERUMO KABUSHIKI KAISHA, Shibuya-ku (JP)

(21) Appl. No.: 14/047,151

(22) Filed: Oct. 7, 2013

Related U.S. Application Data
(63) Continuation of application No. PCT/JP2012/059337, filed on Apr. 5, 2012.

(30) Foreign Application Priority Data
Apr. 7, 2011 (JP) 2011-085717

(51) Int. Cl. A61B 1/005; A61B 19/00; A61B 1/07; A61B 1/06; A61B 17/00; A61B 1/05

(52) U.S. Cl. CPC A61B 1/0057; A61B 17/00234; A61B 1/05; A61B 1/07; A61B 1/0676; A61B 19/5202
USPC 600/110; 606/130; 600/249

(57) ABSTRACT

A medical device configured to permit free-adjustment of the curvature-factor by which the region connected to the distal member provided with the medical-treatment member is to be curved. The medical device includes an elongated body having an opening portion at the distal end, a distal member provided with a medical-treatment member held by the elongated body freely movably in an approaching and separating manner with respect to the opening portion, and an operation member. The operation member includes a first member which is connected to the distal member and which transmits a force, that separately-moves the distal member relatively with respect to the elongated body, to the distal member and a second member connected to the distal member, in which caused by the cooperation between the first member and second member, it is possible to freely-adjust the curvature-factor by which the first member is curved.

### CROSS REFERENCES TO RELATED APPLICATIONS

This application is a continuation of International Application No. PCT/JP2012/059337 filed on Apr. 5, 2012, and claims priority to Japanese Application No. 2011-085717 filed on Apr. 7, 2013, the entire content of both of which is incorporated herein by reference.

### TECHNICAL FIELD

The present invention generally relates to a medical device.

### BACKGROUND DISCUSSION

There exists a known medical device, such as disclosed in Japanese Unexamined Application Publication No. H5-269134, constituted such that a distal member provided with a medical-treatment member is inserted through a guide tube, with a curved tube portion of the guide tube being capable of being curved. For the treatment member, there exist, for example, an ultrasound probe described in Japanese Unexamined Application Publication No. H5-269134, and a camera used for an endoscope or the like. By curving the curved tube portion of the guide tube, it is possible to observe a region in a direction different from the insertion direction by means of the treatment member, to inspect the region, to apply a treatment to the region, and so on.

However, for the medical device disclosed in the Patent Document 1, it is not possible to change the curvature-factor by which the curved tube portion is curved. Since the range in which the curved tube portion can be curved will be restricted, there is such a request that in a more free-range, it is possible to make an observation, to make an inspection, to apply treatment and so on.

### SUMMARY

The medical device makes it possible to freely-adjust the curvature-factor by which the region connected to the distal member provided with the medical-treatment member is to be curved and in which by means of the medical-treatment member provided at the distal member, it is possible to make an observation, to make an inspection, to apply treatment and so on in a more free-range.

According to one aspect, a medical device comprises: an elongated body possessing an open distal end; a distal member movable relative to the elongated body toward and away from the open distal end of the elongated body; a medical-treatment member mounted at the distal member so that the medical-treatment member and the distal member move together as a unit; a cable connected to the medical-treatment member, a portion of the cable being positioned in the elongated body; and a first member connected to the distal member, with a portion of the first member being positioned in the elongated body and movable relative to the elongated body. A stopper is operable from one position permitting movement of the first member relative to the elongated body to an other position preventing movement of the first member relative to the elongated body. A second member is connected to the distal member, with a portion of the second member being positioned in the elongated body and movable relative to the elongated body, and wherein the second member is either the cable or an elongated wire member. An operation lever is connected to the second member so that the operation lever and the second member move together. The operation lever is movable to move the second member when the stopper is in the other position to change a direction toward which the medical-treatment member is directed.

In accordance with another aspect, a medical device includes: an elongated body possessing an open distal end; a distal member provided with a medical-treatment member, with the distal member being held by the elongated body in a freely movable manner in which the distal member moves both toward and away from the open distal end of the elongated body; and an operation member which includes a first member connected to the distal member to transmit a moving force to the distal member to move the distal member relative to the elongated body toward and away from the open distal end of the elongated body, with the first member being configured to be curved so that the first member exhibits a curvature factor. The operation member also includes a second member connected to the distal member, wherein the curvature-factor by which the first member is curved is freely adjustable based on cooperation between the first member and the second member.

The medical device is configured so that it is possible for the operation member to adjust the curvature-factor, by which the first member connected to the distal member is curved, in cooperation of the first member and the second member. Depending on the medical-treatment member provided at the distal member, it is possible to make an observation, to make an inspection, to apply treatment and so on in a more free-range.

It is preferable for the operation member that it is possible to freely-adjust the direction toward which the distal end of the distal member is directed. In this case, it is possible to make an observation, to make an inspection, to apply treatment and so on in a still more free-range.

The operation member preferably adjusts the curvature-factor, by which the first member is curved, by separately-moving the distal member relative to the elongated body and by adjusting the amount of protrusion by which the distal member protrudes distally beyond the open end. In this case, it is possible to rather easily adjust the curvature-factor by which the first member is curved by only adjusting the amount of protrusion of the distal member.

It is preferable for the operation member to curve the first member by separately-moving the distal member by way of the first member relative to the elongated body in a state of restricting the distance between the opening portion and the distal member by way of the second member, or to curve the first member by pulling-down or pulling-in the second member from the distal side in a state of restricting the distance between the opening portion and the distal member by means of the first member. In this case, it is possible for the operation member to curve the first member by a push-out method or by a pull-down method. In other words, in the push-out method, the first member is curved by separately-moving the distal member relative to the elongated body by way of the first member in a state of restricting the distance between the opening portion and the distal member by means of the second member. On the other hand, in the pull-down or pull-in method, the first member is curved by pulling-down the second member from the distal side in a state of restricting the distance between the opening portion and the distal member by way of the first member. In this manner, it is possible to curve the first member according to various methods and it is possible to curve the first member with a preferable angle by employing a method in response to the applying region.

It is preferable that the first member and the second member are both constituted by wire-shaped bodies; and the operation member further includes a first stopper for fixing the first member, and a second stopper for fixing the second member. In this case, even in either method of the push-out method and the pull-down method, it is possible to curve the first member while maintaining the curvature-factor by means of the first stopper or the second stopper. Also, since the first member and second member are constituted by wire-shaped bodies, it is possible to set the diameter size of the mechanism for curving the first member to have a small size. For this reason, it is possible to reduce the diameter of the long-shaped body and it is also possible to achieve a reduced invasiveness.

It is preferable that the treatment member of the distal member is a camera, the wire-shaped body as the first member is a light guide which is attached to the distal member for carrying out illumination when taking an image by the camera, and the wire-shaped body as the second member is a camera cable which is connected to the camera. In this case, it is not necessary for the wire-shaped bodies as the first and second members to use exclusive wires or the like, and it is possible to make the diameter of the long-shaped body smaller and it is possible to achieve the reduced invasiveness much more.

It is preferable that the first member is a tubular body having flexibility which is inserted through the elongated body and which is connected to the distal member, the second member is constituted by a wire-shaped body, and the operation member further comprises a stopper for fixing the long-shaped body with respect to the tubular body. In this case, it is possible to curve the first member while maintaining the curvature-factor by the stopper.

It is preferable that the distal member and the tubular body comprise a lumen allowing insertion of another medical-treatment member therethrough. In this case, it is possible to pull out another medical-treatment member from the distal member connected to the curved first member and it is possible to achieve a reduced invasiveness compared with a case in which a treatment is carried out by using, for example, both of a medical device installed with a camera and a medical device installed with another treatment member simultaneously.

The tubular body can included a recessed portion that is recessed relative to the surrounding portion of the outer circumferential surface and concurrently, includes a long groove extending toward the longitudinal direction; and the wire-shaped body as the second member is housed in a space which is formed between the long groove and the elongated body and is pulled out from the inside of the long groove toward the outside along with the mechanism of curving the first member connected to the distal member which is protruded from the opening portion. In this case, it is possible to carry out the operation of curving the first member 61 without any problem.

It is preferable that the treatment member of the distal member is a camera, and the wire-shaped body as the second member is a camera cable which is connected to the camera. In this case, it is not necessary to use an exclusive-use wire or the like for the wire-shaped body as the second member, it is possible to reduce the diameter of the long-shaped body compared with a case in which an exclusive-use wire is used, and it is possible to achieve a reduced invasiveness.

According to another aspect, a method of changing a direction of orientation of a medical-treatment member comprises: moving a distal member in a forward direction away from an open distal end of an elongated body, wherein the medical-treatment member is mounted in the distal member so that the distal member and the medical-treatment member move together as a unit, wherein a first member is connected to the distal member to transmit a moving force to the distal member to move the distal member relative to the elongated body away from the open distal end of the elongated body, wherein the first member is configured to be curved, and wherein a second member is connected to the distal member. The method also involves, after moving the distal member in the forward direction away from the open distal end of an elongated body, fixing the first member relative to the elongated body so that movement of the distal member relative to the elongated body is prevented, and moving the second member relative to the elongated body while the first member is fixed relative to the elongated body to curve the first member and change the direction toward which the medical-treatment member is directed.

Still other purposes, features and characteristics of the medical device will become more clear by referring to the following detailed description considered with reference to the attached drawings.

### BRIEF DESCRIPTION OF DRAWINGS

FIGS. 1A and 1B are a front view and a plan view respectively of a medical device according to one embodiment disclosed by way of example.

FIG. 2 is a longitudinal cross-section view showing a main portion of the medical device.

FIG. 3A is a cross-sectional view taken along the section line 3A-3A line in FIG. 2.

FIG. 3B is a cross-sectional view taken along the section line 3B-3B line in FIG. 2.

FIG. 3C is a cross-sectional view taken along the section line 3C-3C line in FIG. 2.

FIG. 3D is a cross-sectional view taken along the section line 3D-3D line in FIG. 2.

FIG. 4 is a cross-sectional view to be used for an explanation of a usage example (push-out method) of the medical device in the first embodiment.

FIG. 5 is a cross-sectional view to be used for an explanation of a usage example of the medical device according to the first embodiment.

FIG. 6 is a cross-sectional view to be used for an explanation of a usage example of the medical device according to the first embodiment.

FIG. 7 is a cross-sectional view to be used for an explanation of a usage example of the medical device according to the first embodiment.

FIG. 8 is a cross-sectional view to be used for an explanation of a usage example of the medical device according to the first embodiment.

FIG. 9 is a cross-sectional view to be used for an explanation of a usage example of the medical device according to the first embodiment.

FIG. 10 is a cross-sectional view to be used for an explanation of a usage example of the medical device according to the first embodiment.

FIG. 11 is a cross-sectional view to be used for an explanation of another usage example (pull-down method) of the medical device according to the first embodiment.

FIG. 12 is a cross-sectional view to be used for an explanation of another usage example of the medical device in the first exemplified embodiment;

FIG. 13A-D are cross-sectional views showing a usage example (push-out method) of the medical device according to a second embodiment disclosed by way of example.

FIG. 14A-D are cross-sectional views showing another usage example (pull-down method) of the medical device according to the second embodiment.

FIG. 15 is a cross-sectional view along the section line 15-15 in FIG. 13A.

FIG. 16 is a side view of a medical device according to a third embodiment representing another example of the medical device disclosed here.

FIGS. 17A-17C are schematic views showing an operation example for curving a first member connected to a distal member in the medical device of the third embodiment.

FIG. 18A is a cross-sectional view taken along the section line 18A-18A in FIG. 17A.

FIG. 18B is a cross-sectional view taken along the section line 18B-18B in FIG. 17A.

FIG. 18C is a cross-sectional view taken along the section line 18C-18C in FIG. 17A.

FIG. 18D is a cross-sectional view taken along the section line 18D-18D in FIG. 17A.

### DETAILED DESCRIPTION

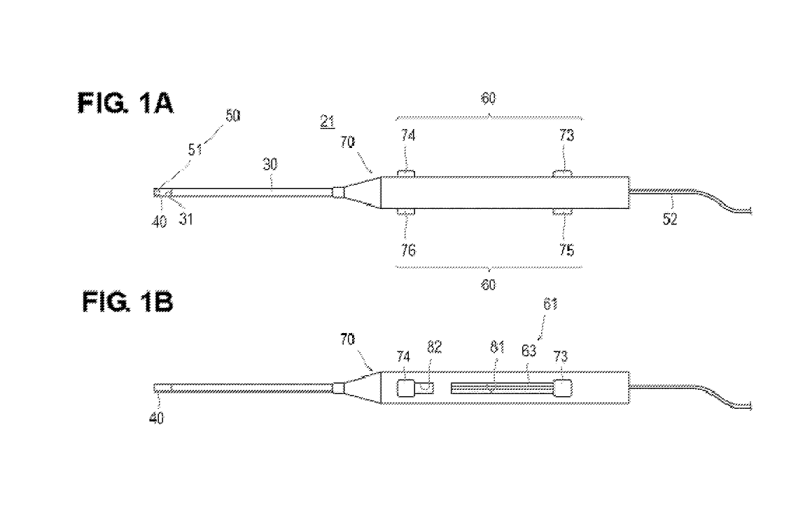

Set forth below is a detailed description of embodiments of a medical device representing examples of the medical device disclosed here. To facilitate the understanding, the drawings include portions which are graphically-illustrated in exaggerated forms and there is a case in which the size ratio is different from the actual ratio. Note that as a matter of convenience of explanation, hereinafter, the right side of each member in FIGS. 1A and 1B will be referred to as the proximal side or proximal end, and the left side as the distal side or distal end for each member.

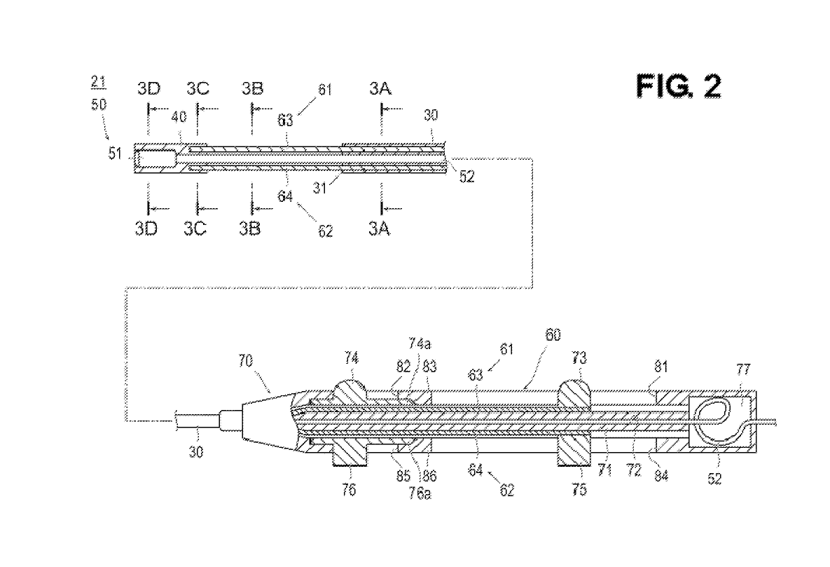

Referring initially to FIGS. 1-3, a medical device 21 according to one embodiment disclosed here includes an elongated (long-shaped) body 30 having an opening portion 31 at the distal end, a distal member 40 held by the elongated body 30 freely movably in an approaching and separating manner with respect to the opening portion 31 and which is provided with a medical-treatment member 50, and an operation member 60. The operation member 60 includes a first member (elongated first member) 61 which is connected to the distal member 40 and which transmits a force, that separately-moves the distal member 40 relatively with respect to the elongated body 30, to the distal member 40 and a second member (elongated second member) 62 connected to the distal member 40, in which caused by the cooperation between the first member 61 and second member 62, it is possible to freely-adjust the curvature-factor (amount or degree of curvature) by which the first member 61 is curved. For the first embodiment, both of the first member 61 and the second member 62 are constituted by wire-shaped or wire bodies, and the operation member 60 further includes a first stopper 74 for fixing the first member 61 and a second stopper 76 for fixing the second member 62. The first member 61 is constituted by a first wire 63 as a wire-shaped body and the second member 62 is constituted by a second wire 64 as a wire-shaped body. The first wire 63 possesses a rigidity by which it is possible to transmit the force to the distal member 40 when the wire 63 is fed or pushed toward the distal side. The medical-treatment member 50 includes a camera 51 for observing the inside of a living body.

Set forth next is a more detailed description of the medical device. The elongated body 30 is to be inserted into a body lumen of a human body. The proximal portion of the elongated body 30 is connected to a hand-side operation unit 70. The hand-side operation unit 70 includes a center shaft portion 71 formed with a center hole 72 and on the upper surface side of the hand-side operation unit 70, there are disposed a first operation lever 73 and the first stopper 74. On the lower surface side of the hand-side operation unit 70, there are disposed a second operation lever 75 and the second stopper 76. A camera cable 52 connected to the camera 51 as the medical-treatment member 50 is inserted-through or passes through the center hole 72 of the center shaft portion 71. The camera cable 52 extends outside the hand-side operation unit 70 by way of a cable housing room 77 provided at the proximal portion of the hand-side operation unit 70. In the cable housing room 77, there is housed the camera cable 52 in a state of being wound in a loop shape.

The first operation lever 73 is connected to the first wire 63 in the operation member 60 and is operated to move the first wire 63. That is, the first operation lever 73 and the first wire 63 move together. On the upper surface of the hand-side operation unit 70, there is formed a longitudinally extending first guide hole 81. The first operation lever 73 is movable in a freely sliding manner along the first guide hole 81. By slide-moving (slidably moving) the first operation lever 73, the first wire 63 moves along the center shaft portion 71. A state in which the first operation lever 73 is in contact with the end surface on the proximal side of the first guide hole 81 corresponds to a backward-limit position or an initial position of the first operation lever 73 and a state in which the first operation lever 73 is in contact with the end surface of the distal side of the first guide hole 81 corresponds to a forward-limit position of the first operation lever 73.

The first stopper 74 is operated to fix the position of the first wire 63. On the upper surface of the hand-side operation unit 70, there is formed a longitudinally extending first locking hole 82. The first stopper 74 is freely slidably movable along the first locking hole 82. The first stopper 74 includes a tongue portion 74a which extends toward the proximal side or in the proximal direction. The hand-side operation unit 70 is provided with a first lock unit 83 which displaces the tongue portion 74a of the first stopper 74, upon being slidably moved toward the proximal end or in the proximal direction, toward the center shaft portion 71. By slide-moving the first stopper 74 toward the proximal side or in the proximal direction, the first wire 63 is sandwiched between the tongue portion 74a of the first stopper 74 and the center shaft portion 71, and the position of the first wire 63 is fixed. The first stopper 74 moves between a fixed position for fixing the position of the first wire 63 (preventing movement of the first wire 63) and an open position for allowing movement of the first wire 63.

The second operation lever 75 is connected to the second wire 64 in the operation member 60 and is operated to move the second wire 64. That is, the second operation lever 75 and the second wire 64 move together. On the lower surface of the hand-side operation unit 70, there is formed a longitudinally extending second guide hole 84. The second operation lever 75 is freely slidably movable along the second guide hole 84. By slide-moving the second operation lever 75, the second wire 64 moves along the center shaft portion 71. A state in which the second operation lever 75 is in contact with the end surface on the proximal end of the second guide hole 84 corresponds to a backward-limit position or an initial position of the second operation lever 75. There is never a case in which the second operation lever 75 is positioned on the distal side beyond the first operation lever 73.

The second stopper 76 is operated to fix the position of the second wire 64. On the lower surface of the hand-side operation unit 70, there is formed a longitudinally extending second locking hole 85. The second stopper 76 is provided freely slide-movably along the second locking hole 85. The second stopper 76 includes a tongue portion 76a which extends toward the proximal side or the proximal direction. The hand-side operation unit 70 is provided with a second lock unit 86 which displaces the tongue portion 76a of the second stopper 76, which was slide-moved (slidably moved) in the proximal direction toward the center shaft portion 71. By slide-moving the second stopper 76 toward the proximal side or in the proximal direction, the second wire 64 is sandwiched between the tongue portion 76a of the second stopper 76 and the center shaft portion 71, and the position of the second wire 64 is fixed. The second stopper 76 moves between a fixed position for fixing the second wire 64 is fixed (preventing movement of the second wire 64) and an open position for allowing the movement of the second wire 64.

The constituent material of the hand-side operation unit 70 can be a thermoplastic resin of polycarbonate, polyamide, polysulfone, polyarylate, methacrylate-butylene-styrene copolymer or the like.

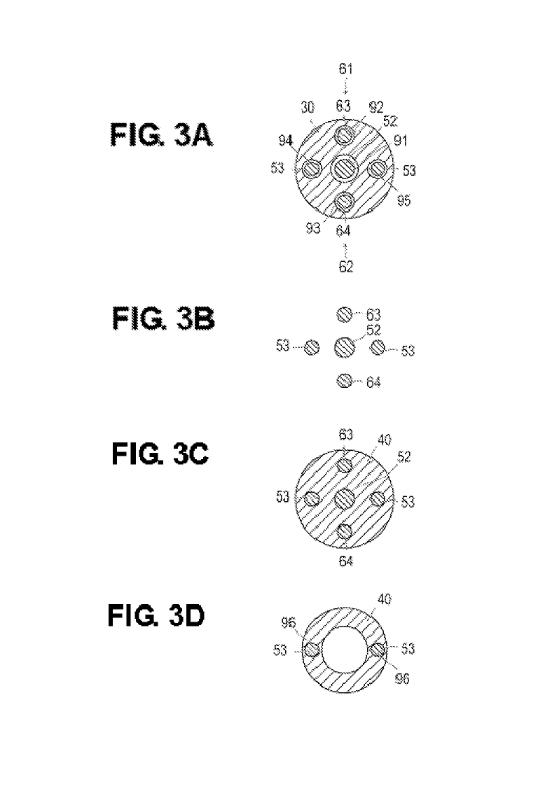

The elongated body 30 includes five (5) separate lumens 91, 92, 93, 94, 05 (see FIG. 3A). The camera cable 52 is positioned in the central lumen 91, the first wire 63 is positioned in the lumen 92 located just-above the central lumen 91, the second wire 64 is positioned in the lumen 93 just-below the central lumen 91, and respective lighting guides 53, 53 are positioned in respective ones of the right and left lumens 94, 95 positioned on diametrically opposite sides of the central lumen 91. The light guides 53 are attached to the distal member 40 to carry out illumination when taking an image by the camera 51.

The constituent material forming the elongated body 30 is preferably a material having flexibility to a certain degree and, for example, it is preferable to employ a thermoplastic resin of polyamide, polyester, polyamide elastomer, polyester elastomer, polyolefin (for example, polyethylene, polypropylene, ethylene-propylene copolymer, ethylene-vinyl acetate copolymer, and cross-linking or partial cross-linking substance thereof), polyvinyl chloride, polyurethane or the like.

The distal member 40 is disposed on the distal side of the elongated body 30 and is provided with the camera 51. The distal member 40 has a tubular shape whose outer diameter is approximately the same as the outer diameter of the elongated body 30. The first wire 63 and the second wire 64 are connected to the proximal end of the distal member 40 (see FIG. 3C). The respective light guides 53 are positioned in and pass through lumens 96 formed in the distal member 40 (see FIG. 3D) and are exposed at the distal surface of the distal member 40. At the position between the opening portion 31 and the distal member 40, the distal member 40 is held on the elongated body 30 by at least the first wire 63. The first wire 63 is moved advancingly and retreatingly (forward and backward) by slide-moving the first operation lever 73, and the second wire 64 is moved advancingly and retreatingly (forward and backward) by slide-moving the second operation lever 75. Thus, the distal member 40 is held on the elongated body 30 freely movably in an approaching and separating manner with respect to the opening portion 31. The light guides 53 also exert a function of holding the distal member 40 on the elongated body 30 in addition to the first wire 63.

The illustrated and described example of the medical device includes 2-pieces of light guides (two light guides) 53, but it is possible to use only a single light guide. Also, in case of the camera provided with integral illumination means, the light guide is not an indispensable element.

The constituent material from which the distal member 40 is made can be a material similar to the constituent material of the elongated body 30 mentioned above.

The operation member 60, as described above, includes the first wire 63 and the second wire 64, and at least the first wire 63 possesses a rigidity by which it is possible to transmit a force, while the first wire 62 is being fed or moved toward the distal end or in the distal direction, to the distal member 40. That is, when pushing the first wire 63 in the forward direction, the first wire 63 is able to transmit the forwardly directed force to the distal member 40 to move the distal member 40 in the distal direction. There is no limitation on the constituent material forming the first wire 63, and it is sufficient if the material constituting the first wire 63 possesses the rigidity by which it is possible to move or feed the distal member 40 toward the distal end. The constituent material of the first wire 63 can be a metal having flexibility, a polymeric material having comparatively high rigidity or a material formed by combining those above appropriately. For example, the metal corresponds to Ni−Ti alloy, stainless steel, Cu—Zn alloy, cobalt alloy or tantalum, and the polymeric material corresponds to polyamide, polyimide, ultra high molecular weight polyethylene, polypropylene, aromatic polyether ketone (for example, PEEK) or fluorine resin. There is also no limitation on the constituent material forming the second wire 64, and it is possible to employ the same material as the constituent material of the first wire 63, and it is also possible to use a constituent material having a weaker rigidity than that of the constituent material of the first wire 63. In this embodiment, the second wire 64 is constituted by a string member having a weaker rigidity than that of the first wire 63. The string member is composed of, for example, a polymeric material.

In the first embodiment, it is possible for the operation method of curving the first wire 63 through operation member 60 to employ two kinds of methods, namely a push-out method and a pull-down method. In case of the push-out method, it is possible to curve the first wire 63 by separately-moving the distal member 40 relatively with respect to the elongated body 30, in other words, by feeding the distal member 40 toward the distal side in a state of restricting the distance between the opening portion 31 and the distal member 40 by the first wire 64. On the other hand, in the case of the pull-down method, it is possible to curve the first wire 63 by pulling-down the second wire 64 from the distal side in a state of restricting the distance between the opening portion 31 and the distal member 40 by the first wire 63.

It is preferable for the operation member 60 that it is possible to freely-adjust the direction toward which the distal end of the distal member 40 is directed. This is because it is possible, in a still more free-range, to make an observation by taking an image by the camera 51, to make an inspection, to apply treatment by being used together with another treatment member and so on. In case of the push-out method, it is possible to change the direction toward which the distal end of the distal member 40 is directed (i.e., to change the orientation of the distal member and the medical-treatment member) by adjusting the amount of feeding of the first wire 63 toward the distal side. On the other hand, in case of the pull-down method, it is possible to change the direction toward which the distal end of the distal member 40 is directed by adjusting the amount of pulling-down of the second wire 64 from the distal side.

The operation member 60 adjusts the curvature-factor, by which the first wire 63 is curved, by separately moving the distal member 40 relative to the elongated body 30 and by adjusting the amount of protrusion by which the distal member 40 protrudes from the opening portion 31. By only adjusting the amount of protrusion of the distal member 40, it is possible to rather easily adjust the curvature-factor by which the first wire 63 is curved. In either case of the push-out method and the pull-down method, by adjusting the amount of feeding of the first wire 63 in a situation before curving the first wire 63 toward the distal side, it is possible to change the amount of protrusion by which the distal member 40 protrudes from the opening portion 31. Depending on this amount of protrusion, in case of the push-out method, it is possible to restrict the distance between the opening portion 31 and the distal member 40 by means of the second wire 64. On the other hand, in case of the pull-down method, it is possible to restrict the distance between the opening portion 31 and the distal member 40 by means of the first wire 63. Thus, the distance between the opening portion 31 and the distal member 40 corresponds to the curvature radius when curving the first wire 63, and the curvature-factor by which the first wire 63 is curved will be determined. For example, when the amount of protrusion by which the distal member 40 protrudes from the opening portion 31 is lengthened or increased, the distance between the opening portion 31 and the distal member 40 becomes longer and the curvature radius when curving the first wire 63 becomes larger or increases, and as a result, the curvature-factor by which the first wire 63 is curved becomes smaller. Conversely, when the amount of protrusion by which the distal member 40 protrudes from the opening portion 31 is shortened or reduced, the distance between the opening portion 31 and the distal member 40 becomes shorter and the curvature radius when curving the first wire 63 becomes smaller, and as a result, the curvature-factor by which the first wire 63 is curved becomes larger.

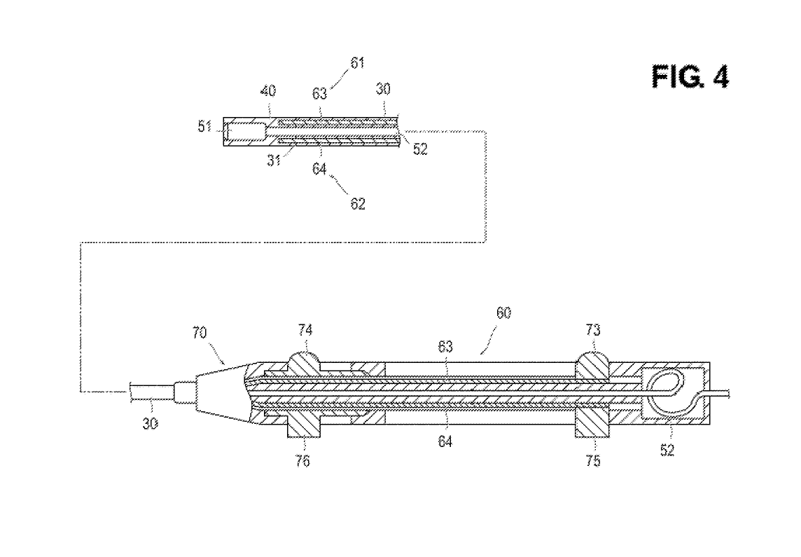

Next, referring to FIGS. 4-10, there will be explained an operation example of curving the first wire 63 by the push-out method with regard to the medical device 21 of the first embodiment. For the sake of easy understanding, FIGS. 4 to 10 show the distal side of the medical device 21 in an enlarged scale compared with the hand-side operation unit 70.

First, as shown in FIG. 4, the first operation lever 73 and the second operation lever 75 are moved backward to the initial positions. The first stopper 74 and the second stopper 76 are moved to the open positions at which the movements of the first and second wires 63, 64 are allowed or permitted. The distal member 40 is moved to be most approached with respect to the elongated body 30. That is, the distal member 40 is moved so that it is closest to the elongated body 30, in contact with elongated body 30 in the illustrated example.

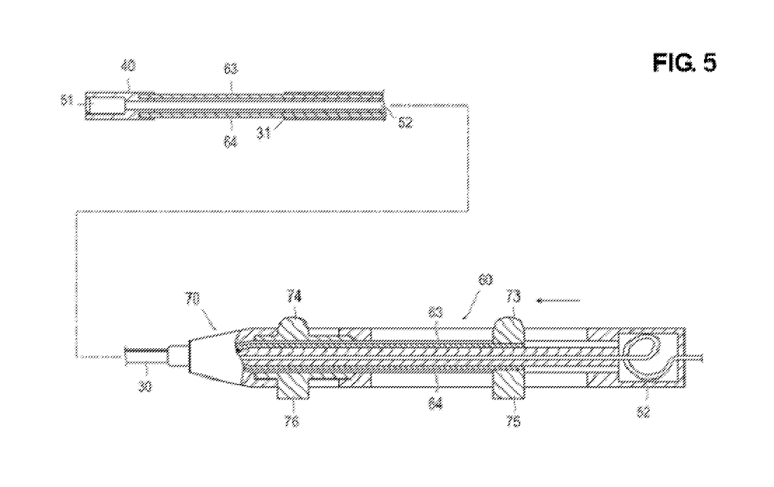

As shown in FIG. 5, the first operation lever 73 is slide-moved from the initial position toward the distal end or in the distal direction. By moving the first operation lever 73 forward, the first wire 63 is fed or moved toward the distal end or in the distal direction. The force when feeding the first wire 63 toward the distal side is transmitted to the distal member 40 by the rigidity of the first wire 63. The distal member 40 protrudes from (extends distally beyond) the opening portion 31 by being separately-moved with respect to the elongated body 30. The amount of protrusion of the distal member 40 is determined in response to the amount of movement of the first operation lever 73. The amount of protrusion of the distal member 40 is adjusted by moving the first operation lever 73 advancingly and retreatingly (forward and backward). It is not necessary to slide-move the second operation lever 75. This is because also the second wire 64 connected to the distal member 40 will be moved together with the movement of the distal member 40.

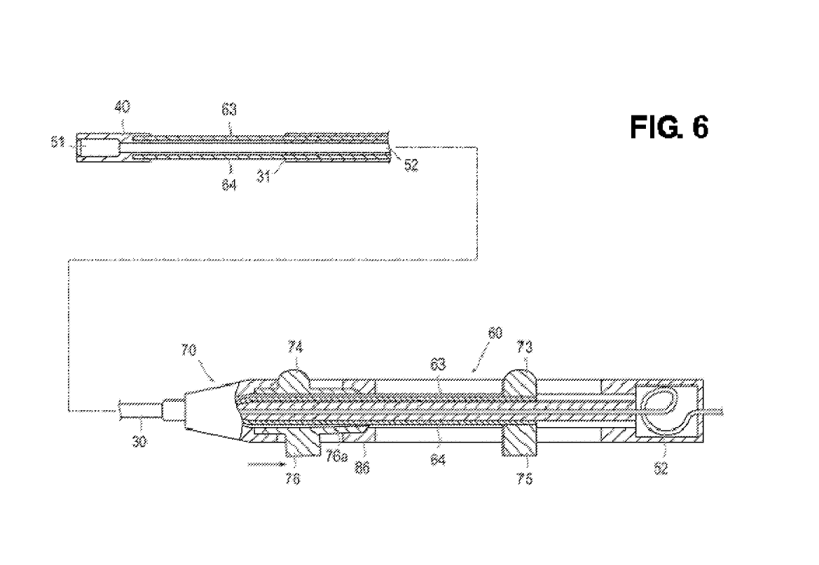

As shown in FIG. 6, in the push-out method, the second stopper 76 is slide-moved to a fixed position and the second wire 64 is fixed. By virtue of the fixed second wire 64, the distance between the opening portion 31 and the distal member 40 is restricted.

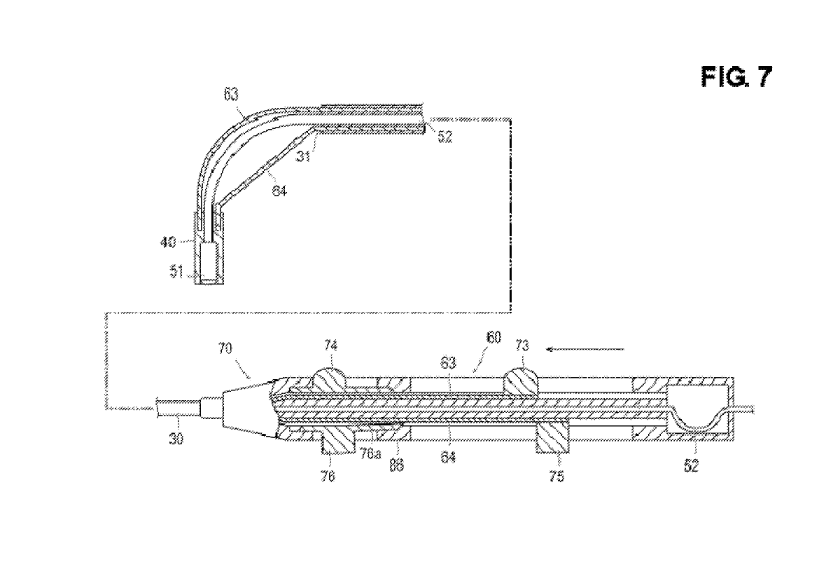

As shown in FIG. 7, in the push-out method, the first operation lever 73 is moved forward and the first wire 63 is fed or moved in the distal direction toward the distal end in a state in which the distance between the opening portion 31 and the distal member 40 is restricted by the second wire 64. Thus, the first wire 63 begins to curve. A situation thus arises in which the distance between the opening portion 31 and the distal member 40 corresponds to the curvature radius when curving the first wire 63, and the curvature-factor by which the first wire 63 is curved is determined.

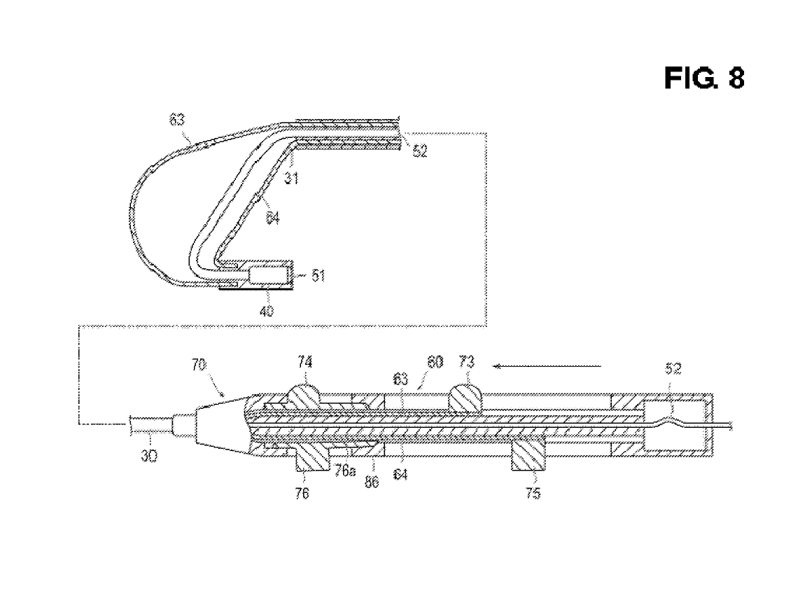

As shown in FIG. 8, while keeping a state of restricting the distance between the opening portion 31 and the distal member 40 by means of the second wire 64, the first operation lever 73 is further moved forward and the first wire 63 is further fed toward the distal side. In case of the push-out method, by adjusting the amount of feeding or forward movement of the first wire 63 toward the distal side, it is possible to change the direction toward which the distal end of the distal member 40 is directed. In FIG. 7, the distal end of the distal member 40 is directed downward in the drawing so that the distal member 40 is directed in a direction at an angle of 90 degrees with respect to the insertion direction. On the other hand, in FIG. 8, the distal end of the distal member 40 is directed to the right side in the drawing so that the distal member 40 is directed in a direction at an angle of 180 degrees with respect to the insertion direction. In this manner, it is possible for the operation member 60 to freely-adjust the direction toward which the distal end of the distal member 40 is directed. Thus, it is possible, in a still more free-range, to make an observation by taking an image by the camera 51, to make an inspection, to apply treatment by being used together with another treatment member and so on.

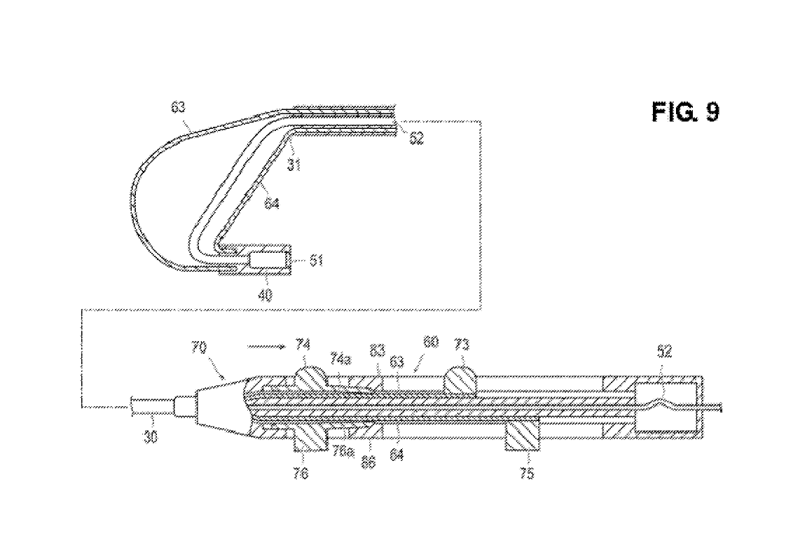

As shown in FIG. 9, slidably moving the first stopper 74 in the proximal direction to a fixed position, the first wire 63 is fixed, and the posture of the distal member 40 is maintained. Thus, the curving operation of the first wire 63 in the medical device 21 is finished.

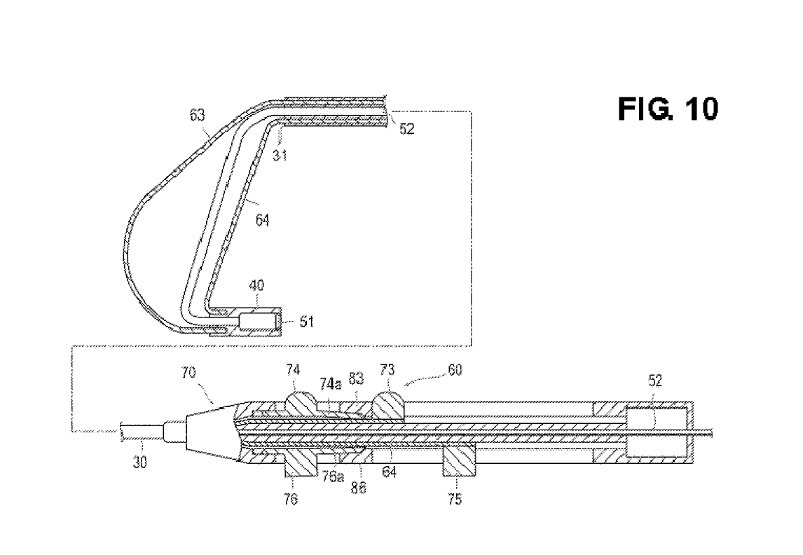

As shown in FIG. 10, in a case in which the curvature-factor by which the first wire 63 is curved is desired to be reduced, the amount of protrusion by which the distal member 40 protrudes from the opening portion 31 is lengthened or increased and so the result is that the curvature radius when curving the first wire 63 is lengthened. In FIG. 10, the first operation lever 73 is moved forward as far as the forward-limit position. Although the illustration is omitted, in a case in which conversely, the curvature-factor by which the first wire 63 is curved is desired to be enlarged, it is sufficient if the curvature radius when curving the first wire 63 is to be reduced by reducing the amount of protrusion by which the distal member 40 protrudes from the opening portion 31 and by shortening the distance between the opening portion 31 and the distal member 40.

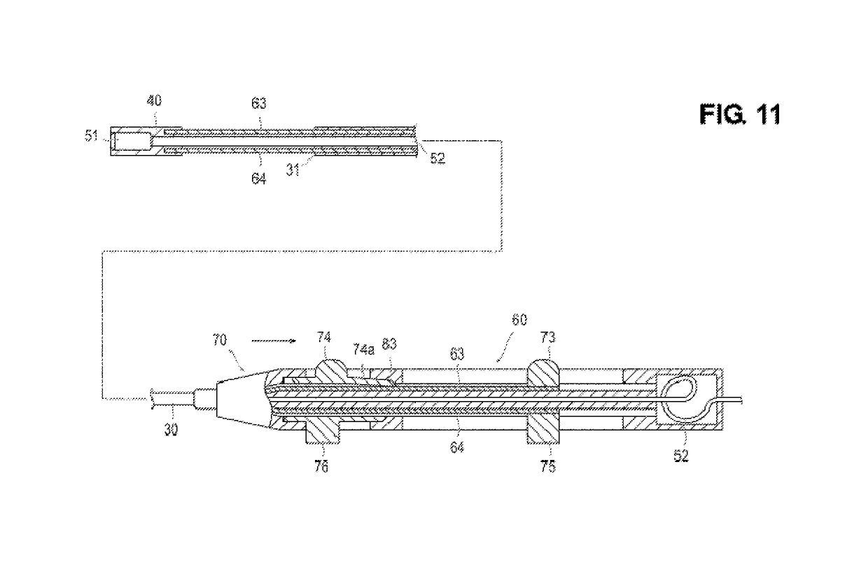

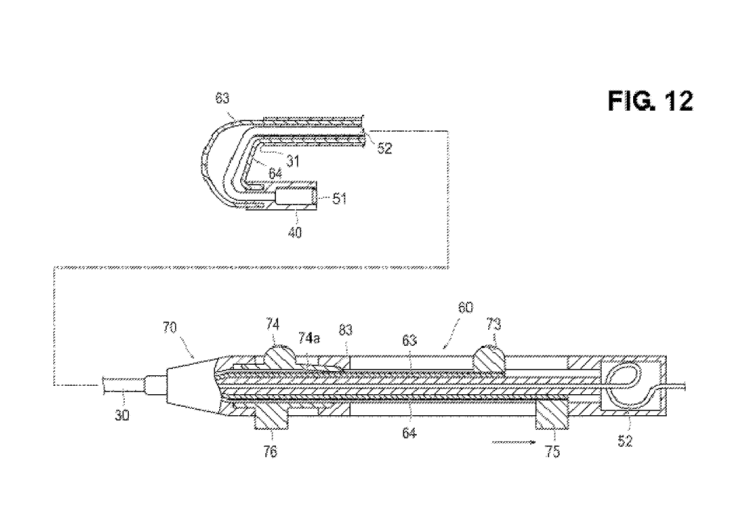

Next, there will be explained an operation example of curving the first wire 63 by the pull-down (pull-in) method with regard to the medical device 21 of the first embodiment with reference to FIGS. 11 and 12. For the sake of easy understanding, FIGS. 11 and 12 show the distal side of the medical device 21 enlarged compared with the hand-side operation unit 70.

As shown in FIG. 11, after adjusting the amount of protrusion of the distal member 40 by moving the first operation lever 73 advancingly and retreatingly (forward and backward), in the pull-down (pull-in) method, the first stopper 74 is slide-moved to a fixed position and the first wire 63 is fixed. By means of the fixed first wire 63, the distance between the opening portion 31 and the distal member 40 is restricted or fixed.

As shown in FIG. 12, in the pull-down method, the second operation lever 75 is moved backward and the second wire 64 is pulled-down from the distal end in a state in which the distance between the opening portion 31 and the distal member 40 is restricted or fixed by means of the first wire 63. Thus, the first wire 63 begins to curve. Also for the pull-down method, the distance between the opening portion 31 and the distal member 40 corresponds to the curvature radius when curving the first wire 63, and the curvature-factor by which the first wire 63 is curved is determined.

While keeping a state of restricting the distance between the opening portion 31 and the distal member 40 by means of the first wire 63, the second operation lever 75 is further moved backward and the second wire 64 is further pulled-down (pulled-in) from the distal end. In case of the pull-down method, by adjusting the amount of pulling-down the second wire 64 from the distal side, it is possible to change the direction toward which the distal end of the distal member 40 is directed. In FIG. 12, the distal end of the distal member 40 is directed to the right side in the drawing, and there is formed an angle of 180 degrees with respect to the insertion direction. In this manner, also for the pull-down method, it is possible for the operation member 60 to freely-adjust the direction toward which the distal end of the distal member 40 is directed. Thus, it is possible, in a still more free-range, to make an observation by taking an image by the camera 51, to make an inspection, to apply treatment by being used together with another treatment member and so on.

The second stopper 76 can then be slide-moved to a fixed position to fix the second wire 64, whereby the posture of the distal member 40 is maintained. Thus, the curving operation of the first wire 63 in the medical device 21 is finished.

It is possible to arbitrarily determine whether the push-out method is to be employed or the pull-down (pull-in) method is to be employed for the operation of curving the first wire 63 in correspondence with the applying region. Compared with the push-out method, it is possible for the pull-down method to curve the first wire 63 more speedily and the sensitivity is better.

As mentioned above, according to the medical device 21 of the first embodiment, it is possible for the operation member 60 to adjust the curvature-factor, by which the first member 61 connected to the distal member 40 is curved, in cooperation of the first member 61 and the second member 62, and for this reason, depending on the medical-treatment member 50 provided at the distal member 40, it is possible to use the medical device to make an observation, to make an inspection, to apply treatment and so on in a more free-range.

It is possible for the operation member 60 to relatively freely-adjust the direction toward which the distal end of the distal member 40 is directed, so that it is possible to use the treatment member 50 in a still more free-range.

The operation member 60 adjusts the curvature-factor, by which the first member 61 is curved, by adjusting the amount of protrusion by which the distal member 40 protrudes from the opening portion 31, and by only adjusting the amount of protrusion of the distal member 40, it is possible to rather easily adjust the curvature-factor by which the first member 61 is curved.

There is employed a configuration in which the operation member 60 to curve the first member 61 by the push-out method or by the pull-down method. In other words, in the push-out method, the first member 61 is curved by separately-moving the distal member 40 relative to the elongated body 30 by means of the first member 61 in a state of restricting the distance between the opening portion 31 and the distal member 40 by means of the second member 62. On the other hand, in the pull-down or pull-in method, the first member 61 is curved by pulling-down (pulling-in) the second member 62 from the distal end in a state of restricting the distance between the opening portion 31 and the distal member 40 by means of the first member 61. In this manner, it is possible to curve the first member 61 according to various methods and it is possible to curve the first member 61 with the preferable angle by employing a method corresponding to the region to be applied.

The first member 61 and the second member 62 are both constituted by wire-shaped bodies, and the operation member 60 further includes the first stopper 74 for fixing the first member 61 and the second stopper 76 for fixing the second member 62, so that even in either method of the push-out method and the pull-down method, it is possible to curve the first member 61 by the first stopper 74 or the second stopper 76 while maintaining the curvature-factor. Also, the first member 61 and the second member 62 are constituted by the first and second wires 63, 64 which have wire-shaped bodies, so that it is possible to set the diameter size of the mechanism for curving the first member 61 to have a small size. Consequently it is possible to reduce the diameter of the elongated body 30 and it is possible to achieve a reduced invasiveness.

It is preferable for the medical device 21 of the first embodiment described above to be applied to a transvaginal endoscope. More specifically, for the laparoscopic operation of inserting an endoscope by way of an abdominal wall, there are inserted an endoscope, a forceps which carries out the operation and the treatment of an internal-organ, and the like. For these endoscope and forceps, there are used materials having rigid properties from the reason that they are rather easily operated. Consequently, in case of carrying out observation or treatment of the rear side of an internal-organ during the operation, it is necessary to reverse, to uplift the internal-organ by the forceps, and so on. On the other hand, for the transvaginal endoscope which is inserted by a transvaginal approach, only a camera is inserted for the reason that the insertion region is narrow and in order to make the invasion smaller. Consequently, even if it is possible to observe the front internal-organ, it is difficult to recognize the whole thereof. In addition, the internal-organ at the periphery of the uterus has various sizes from a large size to a small size, and for this reason, there has been a demand for an endoscope which can be curved in conformity with the shape of the internal-organ. The medical device 21 is an endoscope which can curve the curved portion by changing the radius thereof arbitrarily, so that it is possible to move the observation portion of the distal end of the camera along the internal-organ from a thin portion such as a fallopian tube to a large portion such as a uterus. Therefore, the medical device 21 becomes a preferable device for being applied to the transvaginal endoscope.

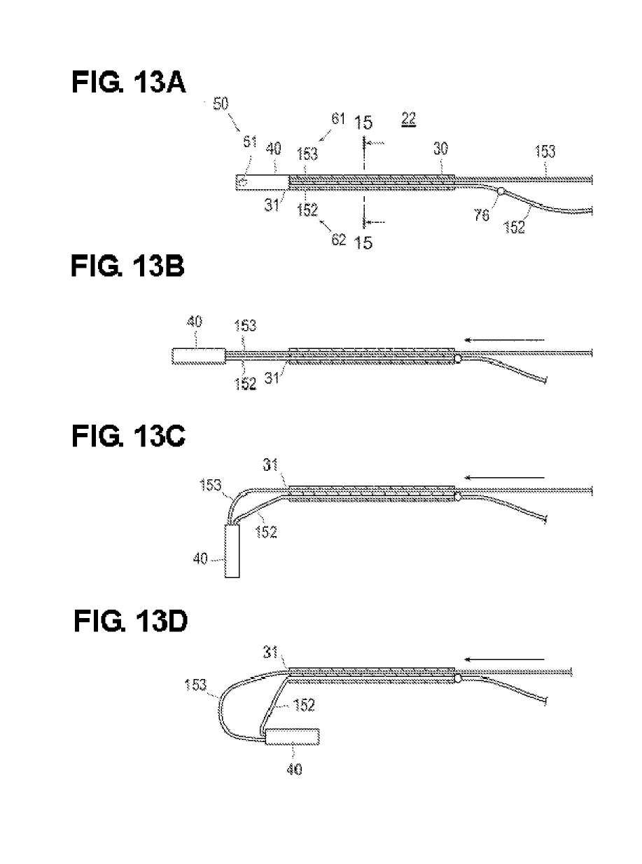

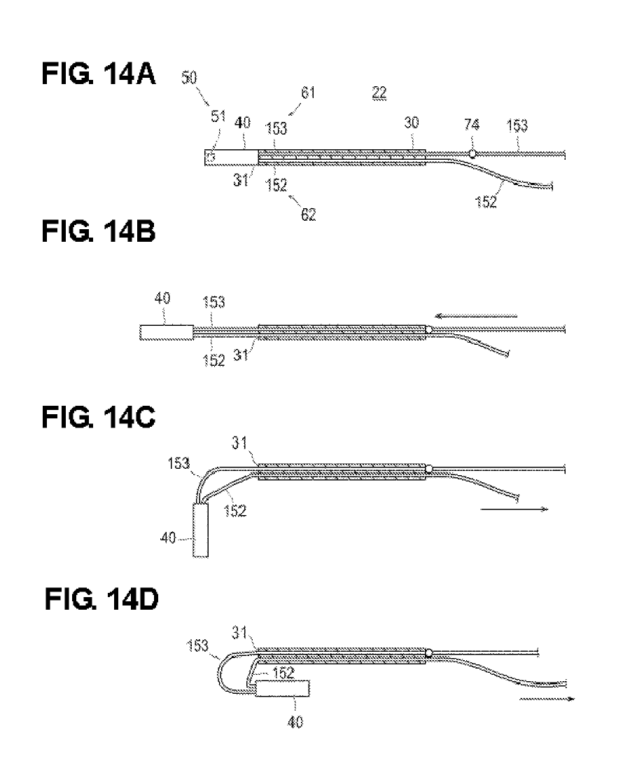

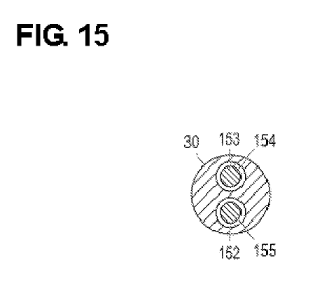

FIGS. 13A to 13D are schematic views showing operation examples of curving the first member 61 by the push-out method in a medical device 22 of a second embodiment, FIGS. 14A to 14D are schematic views showing operation examples of curving the first member 61 by the pull-down or pull-in method in the medical device 22 of the second embodiment. In FIGS. 13A-D and FIGS. 14A-D, the illustration of the hand-side operation unit 70 is omitted for the sake of simplicity, but it is to be understood that the hand-side operation unit 70 is constituted similar to that of the first embodiment. The stopper 76 is schematically shown in FIGS. 13A-D, and the stopper 74 is schematically shown in FIGS. 14A-D. Also, FIG. 15 is a cross-sectional view along the section line 15-15 in FIG. 13A.

In a manner similar to the first embodiment, the medical device 22 according to the second embodiment includes the elongated body 30, the distal member 40 and the operation member 60, in which the first member 61 and the second member 62 of the operation member 60 are both constituted by wire-shaped bodies. In this example, the medical treatment member 50 is in the form of the camera 51.

However, for the second embodiment, the wire-shaped body as the first member 61 is constituted by a light guide 153 which is attached to the distal member 40 in order to carry out the illumination when taking an image by the camera 51, and the wire-shaped body as the second member 62 is constituted by a camera cable 152 which is connected to the camera 51. In this point of view, a difference exists from the first embodiment in which separately from the light guide 53 and the camera cable 52, there is used the first wire 63 for the wire-shaped body as the first member 61 and there is used the second wire 64 for the wire-shaped body as the second member 62. The light guide 153 has rigidity by which it is possible to transmit the force to the distal member 40 when the light guide is fed or moved toward the distal end. Hereinafter, a detailed explanation will be carried out.

The elongated body 30 includes two lumens 154, 155 as shown in FIG. 15. The light guide 153 is positioned in the lumen 154 at the upper portion while the camera cable 152 is positioned in the lumen 155 at the lower portion.

In the medical device 22 of the second embodiment, as shown in FIGS. 13A and 13B, the first operation lever 73 is slide-moved from the initial position toward the distal end in order to curve the light guide 153 by the push-out method. By moving the first operation lever 73 forward, the light guide 153 is fed toward the distal side. The force when feeding the light guide 153 toward the distal side is transmitted to the distal member 40 by the rigidity of the light guide 153. The distal member 40 protrudes from (extends distally beyond) the opening portion 31 by being separately-moved with respect to the elongated body 30. For the push-out method, the second stopper 76 is slide-moved to a fixed position and the camera cable 152 is fixed (prevented from moving). By means of the fixed camera cable 152, the distance between the opening portion 31 and the distal member 40 is restricted.

As shown in FIG. 13C, in the push-out method, the first operation lever 73 is moved forward and the light guide 153 is fed toward the distal end in a state of restricting the distance between the opening portion 31 and the distal member 40 by the camera cable 152. Thus, the light guide 153 begins to curve. The distance between the opening portion 31 and the distal member 40 corresponds to the curvature radius when curving the light guide 153, and the curvature-factor by which the light guide 153 is curved is determined.

As shown in FIG. 13D, while keeping a state of restricting the distance between the opening portion 31 and the distal member 40 by means of the light guide 152, the first operation lever 73 is further moved forward and the light guide 153 is further fed toward the distal end. In case of the push-out method, by adjusting the amount of feeding of the light guide 153 toward the distal end or in the distal direction, it is possible to change the direction toward which the distal end of the distal member 40 is directed.

In the medical device 22 of the second embodiment, in order to curve the light guide 153 by the pull-down or pull-in method, as shown in FIG. 14A and FIG. 14B, the first operation lever 73 is moved advancingly and retreatingly (forward and backward) and the amount of protrusion of the distal member 40 is adjusted. Thereafter, for the pull-down method, the first stopper 74 is slide-moved to a fixed position and the light guide 153 is fixed. By means of the fixed light guide 153, the distance between the opening portion 31 and the distal member 40 is restricted.

As shown in FIG. 14C, in the pull-down method, in a state of restricting the distance between the opening portion 31 and the distal member 40 by the light guide 153, the second operation lever 75 is moved backward and the camera cable 152 is pulled-down from the distal side. Thus, the light guide 153 begins to curve. Also for the pull-down method, the distance between the opening portion 31 and the distal member 40 corresponds to the curvature radius when curving the light guide 153, and the curvature-factor by which the light guide 153 is curved will be determined.

As shown in FIG. 14D, while keeping a state of restricting the distance between the opening portion 31 and the distal member 40 by means of the light guide 153, the second operation lever 75 is further moved backward and the camera cable 152 is further pulled-down from the distal side. In case of the pull-down method, by adjusting the amount of pulling-down the camera cable 152 from the distal side, it is possible to change the direction toward which the distal end of the distal member 40 is directed.

According to the second embodiment, from the fact that the wire-shaped body as the first member 61 of the operation member 60 is constituted by the light guide 153 and the wire-shaped body as the second member 62 is constituted by the camera cable 152, it is not necessary to use exclusive-use wires for the wire-shaped bodies as the first and second members 61, 62. Consequently, it is possible to reduce the diameter of the elongated body 30 compared with that of the first embodiment and it is possible to achieve a still-more reduced invasiveness.

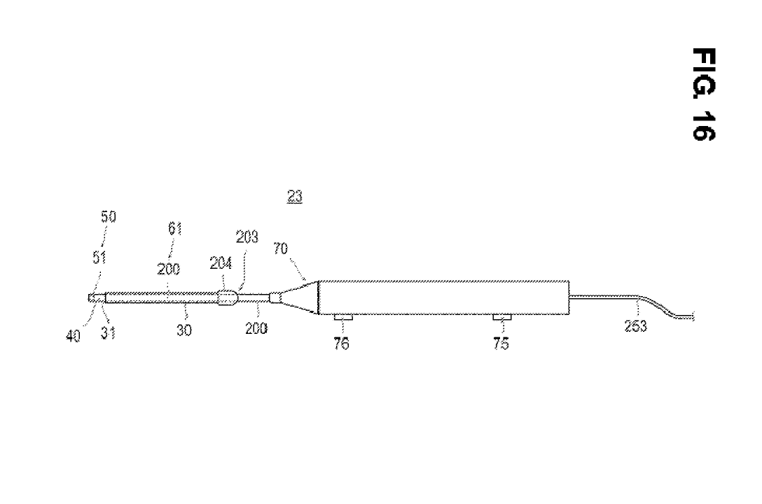

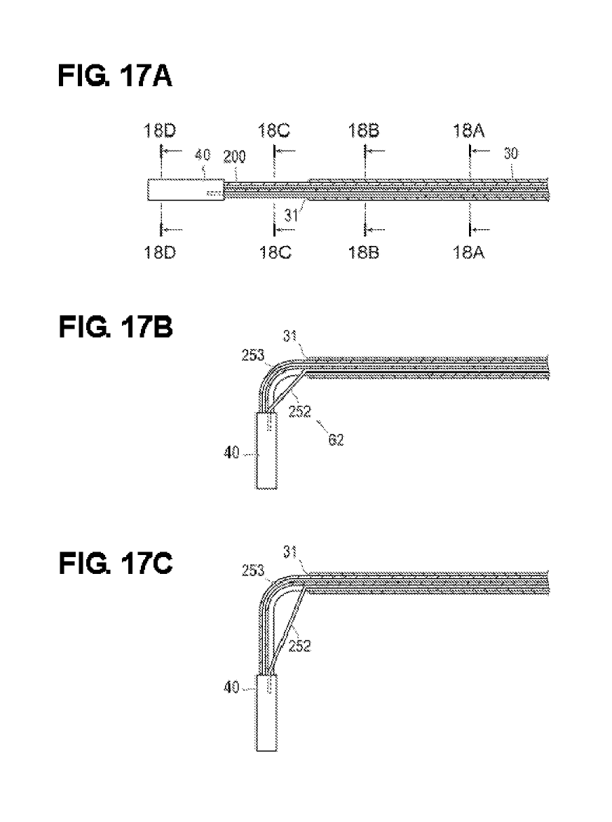

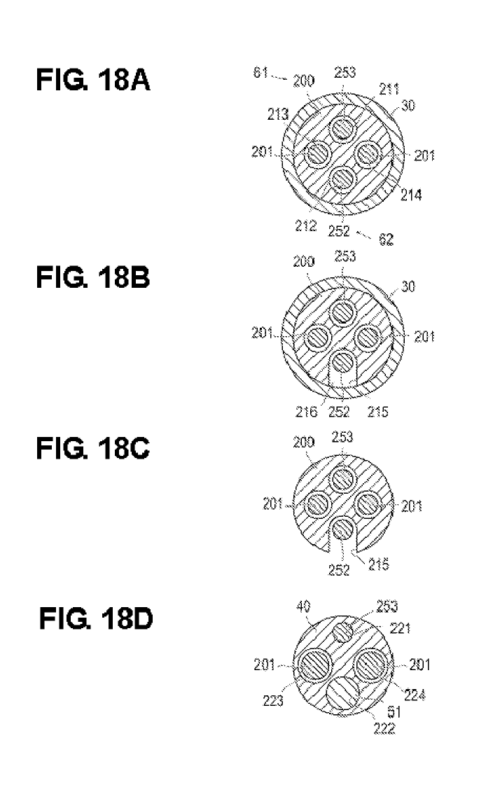

FIG. 16 is a side view showing a medical device 23 according to a third embodiment, FIGS. 17A to 17C are schematic views showing an operation example for curving the first member 61 by the pull-down or pull-in method in the medical device 23 of the third embodiment, and FIGS. 18A-18D are cross-sectional views along the section lines 18A-18A, 18B-18B, 18C-18C, and 18D-18D in FIG. 17A.

The medical device 23 of the third embodiment, similarly to the medical devices in the first and second embodiments, includes the elongated body 30, the distal member 40 and the operation member 60. The operation member 60 includes a first member 61 which is connected to the distal member 40 and which transmits a force, that separately-moves the distal member 40 relative to the elongated body 30, to the distal member 40 and a second member 62 connected to the distal member 40, in which caused by the cooperation between the first member 61 and second member 62, it is possible to freely-adjust the curvature-factor by which the first member 61 is curved. The medical-treatment member 50 is in the form of a camera 51 for observing the inside of a living body.

In this third embodiment, the first member 61 is constituted by a tubular body 200 having flexibility, which is inserted through or positioned in the elongated body 30 and which is connected to the distal member 40; the second member 62 is constituted by a wire-shaped body; and the operation member 60 further includes a stopper 203 for fixing the elongated body 30 with respect to the tubular body 200. In this point of view, the third embodiment is different from the first and second embodiments in which both of the first member 61 and the second member 62 are constituted by the wire-shaped bodies. The wire-shaped body as the second member 62 is, similar to that of the second embodiment, constituted by a camera cable 252 which is connected to the camera 51.

It is sufficient if the operation member 60 is a member which can adjust the curvature-factor, by which the tubular body 200 is curved, by separately-moving the distal member 40 relative to the elongated body 30 and by adjusting the amount of protrusion by which the distal member 40 protrudes from the opening portion 31. In the third embodiment, the operation member 60 is constituted so as to separately-move the elongated body 30 with respect to the distal member 40. Also in this point of view, the third embodiment is different from the first and second embodiments which are constituted so as to separately-move the distal member 40 with respect to the elongated body 30. Hereinafter, a detailed explanation will be set forth.

With reference to FIG. 16, the proximal portion of the tubular body 200 is connected to the hand-side operation unit 70 and the elongated body 30 is freely movable along the tubular body 200. In the first and second embodiments, the distal member 40 is moved toward the distal side, but in the third embodiment, the proximal portion of the tubular body 200 is fixed at the hand-side operation unit 70, so that it is not possible for the distal member 40 to be moved toward the distal side. With regard to the tubular body 200, the distal portion of the tubular body 200 is connected to the distal member 40 and the proximal portion of the tubular body 200 is connected to the hand-side operation unit 70. By the configuration in which the tubular body 200 has rigidity by which the elongated body 30 can be held freely movable, it is possible for the distal member 40 to be separately-moved relative to the elongated body 30 by moving the elongated body 30. Therefore, it can be said that the tubular body 200 transmits the force, for separately-moving the distal member 40 relative to the elongated body 30, to the distal member 40 by using the rigidity of the tubular body 200.

There is no specific limitation on the constituent material forming the tubular body 200. It is sufficient if the material has rigidity by which the elongated body 30 can be held freely movably and concurrently, has flexibility. The constituent material of the tubular body 200 can be, for example, a polymeric material having comparatively high rigidity, which has flexibility. For example, the polymeric material is made of polyamide, polyimide, ultra-high molecular-weight polyethylene, polypropylene, aromatic polyether ketone (for example, PEEK) or fluorine resin.

The stopper 203 maintains a state in which the distance between the opening portion 31 and the distal member 40 is restricted by the tubular body 200 as the first member 61. The stopper 203 includes, for example, a cap portion 204 of a screw type, which is connected to the elongated body 30, in which by screwing-in or rotating the cap portion 204 and thereby pressing a portion of the elongated body 30 inward and toward the diameter direction, the elongated body 30 is fixed with respect to the tubular body 200. Since the distal member 40 is not moved, the first operation lever 73 is not provided at the hand-side operation unit 70. The second operation lever 75 and the second stopper 76 are constituted or configured similar to those of the first and second embodiments.

With reference to FIGS. 18A to 18D, the distal member 40 and the tubular body 200 include lumens 213, 214, 223, 224 for inserting other medical-treatment members 201 through the lumens. It is possible to pull out other medical-treatment members 201 from the distal member 40 with which the curved tubular body 200 is connected, and in comparison with a case in which the treatment is carried out by using both of the medical device installed with the camera and the medical device installed with other treatment member concurrently, it is possible to achieve a reduced invasiveness.

As shown in FIG. 18A, the tubular body 200 includes four lumens 211, 212, 213, 214. A light guide 253 is positioned in the lumen 211 located toward the top side in FIG. 18A, the camera cable 252 is positioned in the lumen 212 located toward the bottom side in FIG. 18A, and other medical-treatment members 201 (for example, needle member, cutting member and the like) are positioned in respective ones of the lumens 213, 214 on the right and left diametrically opposite sides in FIG. 18A.

As shown in FIGS. 18B and 18C, in the tubular body 200, the region adjacent to the distal member 40 changes from the shape of the lumen 212 to have a shape of a long groove 215. The long groove 215 is formed by being sunk or recessed from the outer circumferential surface of the tubular body and concurrently, extends longitudinally.

The camera cable 252 which is a wire-shaped body as the second member 62 is housed in the space 216 which is formed between the long groove 215 and the elongated body 30 (FIG. 18B). When the tubular body 200 is exposed by separately-moving the elongated body 30 with respect to the distal member 40, the space 216 of the long groove 215 is exposed toward the outside (FIG. 18C). Therefore, when curving the tubular body 200 connected to the distal member 40 which protrudes distally from the opening portion 31, a state occurs in which the camera cable 252 is to be pulled out toward the outside from the inside of the long groove 215 (FIGS. 17B and 17C).

As shown in FIG. 18D, the distal member 40 is formed with lumens 223, 224 which communicate with the lumens 213, 214 respectively. Other medical-treatment members 201 are inserted through or positioned in respective ones of the lumens 223, 224. The light guide 253 is fixed at the lumen 221 on the upper side, and the camera 51 connected with the camera cable 252 is fixed at the lumen 222 on the lower side.

In order to curve the tubular body 200 for the medical device 23 of the third embodiment, the elongated body 30 is separately-moved with respect to the distal member 40 while the cap portion 204 of the stopper 203 is loosened, and the elongated body 30 is fixed with respect to the tubular body 200 by screwing-in the cap portion 204 again (FIG. 17A). By the tubular body 200 exposed from the elongated body 30, the distance between the opening portion 31 and the distal member 40 is determined. The stopper 203 maintains a state in which the distance between the opening portion 31 and the distal member 40 is restricted by the tubular body 200.

As shown in FIG. 17B, in a state of restricting the distance between the opening portion 31 and the distal member 40 by the exposed tubular body 200, the second operation lever 75 is moved backward and the camera cable 252 is pulled-down from the distal side. Thus, the camera cable 252 is pulled out from the long groove 215 and the tubular body 200 begins to curve. The distance between the opening portion 31 and the distal member 40 corresponds to the curvature radius when curving the tubular body 200, and there is determined the curvature-factor by which the tubular body 200 is curved. Along with the mechanism of curving the tubular body 200 connected to the distal member 40 which protrudes from the opening portion 31, the camera cable 252 is pulled out from the long groove 215 inside to the outside.

As shown in FIG. 17C, in a case in which the curvature-factor by which the tubular body 200 is curved is desired to be reduced, it is sufficient if the elongated body 30 is separately-moved with respect to the distal member 40, the amount of protrusion by which the distal member 40 protrudes from the opening portion 31 is lengthened, the distance between the opening portion 31 and the distal member 40 is lengthened, and the curvature radius when curving the tubular body 200 is lengthened. Also in this case, the camera cable 252 is pulled out from the inside of the long groove 215 to the outside along with the mechanism of curving the tubular body 200.

As mentioned above, also by the medical device 23 of the third embodiment, similar to that of the first and second embodiments, it is possible to make an observation, to make an inspection, to apply treatment and so on in a more free-range depending on the medical-treatment member 50 provided at the distal member 40 based on the fact that the operation member 60 can adjust the curvature-factor, by which the first member 61 connected to the distal member 40 is curved, depending on the cooperation between the first member 61 and the second member 62.

The operation member 60 includes the first member 61 which transmits a force for separately-moving the distal member 40 relative to the elongated body 30 toward the distal member 40 and the second member 62 connected to the distal member 40, and there is employed a configuration in which the operation member 60 curves the first member 61 by the pull-down or pull-in method. In the pull-down method, it is possible for the first member 61 to be curved by pulling-down the second member 62 from the distal end in a state of restricting the distance between the opening portion 31 and the distal member 40 by means of the first member 61.

The first member 61 is constituted by the tubular body 200 having flexibility, which is inserted through the elongated body 30 and which is connected to the distal member 40; the second member 62 is constituted by the wire-shaped body; and the operation member 60 further includes the stopper 203 for fixing the elongated body 30 with respect to the tubular body 200, so that it is possible to curve the first member 61 while maintaining the curvature-factor by the stopper 203.

The distal member 40 and the tubular body 200 include the lumens 213, 214, 223, 224 for inserting other medical-treatment members 201 therethrough, so that it is possible to pull out other medical-treatment members 201 from the distal member 40 which is connected to the curved first member 61, and in comparison with a case in which the treatment is carried out by using both of the medical device installed with the camera and the medical device installed with other treatment member concurrently, it is possible to achieve a reduced invasiveness.

The tubular body 200 is formed by being sunk or recessed from the surrounding outer circumferential surface of the tubular body 200 and concurrently, includes the long groove 215 extended in the longitudinal direction; the wire-shaped body as the second member 62 is housed in the space 216 formed between the long groove 215 and the elongated body 30, and is pulled out from the inside of the long groove 215 toward the outside along with the mechanism of curving the first member 61 connected to the distal member 40 which protrudes from the opening portion 31. Consequently, it is possible to carry out the operation of curving the first member 61 without any problem.

From the fact that the treatment member 50 of the distal member 40 is constituted by the camera 51 and the wire-shaped body as the second member 62 is constituted by the camera cable 252 connected to the camera 51, it is not necessary to use an exclusive-use wire for the wire-shaped body as the second member 62. Consequently, it is possible to reduce the diameter of the elongated body 30 compared with that in case of using an exclusive-use wire, and it is possible to achieve a reduced invasiveness.

The description above describes the medical devices 21, 22, 23 representing examples of the medical device disclosed here. But the present invention is not limited by those embodiments, as it is possible for each portion constituting the medical device 21, 22, 23 to be replaced by an element having a different constitution or configuration which can exert a similar function. It also possible to add additional features.

For example, there was explained a third embodiment in which the operation member 60 curves the tubular body 200 only by the pull-down or pull-in method, but the tubular body 200 is provided freely movable and besides, by fixing the proximal portion of the elongated body by being connected to the hand-side operation unit, it is possible for the operation member 60, similar to those of the first and second embodiments, to curve the tubular body 200 by the push-out method or by the pull-down method.

Also, it is also possible to employ a constitution or configuration in which the operation member 60 will curve the first member 61 only by the push-out method.

It is also possible to employ a configuration in which the operator curves the first member 61 by fixing the movement of the first member 61 and the second member 62 without providing the first stopper 74, the second stopper 76 and the stopper 203. In this case, it is possible to carry out the fine-adjustment of its curvature-factor more speedily while curving the first member 61.

The detailed description above describes features, characteristics and operational aspects of embodiments of a medical device representing examples of the medical device disclosed here. The disclosure and the present invention are not limited, however, to the precise embodiments and variations described. Various changes, modifications and equivalents could be effected by one skilled in the art without departing from the spirit and scope of the disclosure as defined in the appended claims. It is expressly intended that all such changes, modifications and equivalents which fall within the scope of the claims are embraced by the claims.

What is claimed is:

1. A medical device comprising:
an elongated body possessing an open distal end;
a distal member movable relative to the elongated body toward and away from the open distal end of the elongated body;
a medical-treatment member mounted at the distal member so that the medical-treatment member and the distal member move together as a unit;
a cable connected to the medical-treatment member, a portion of the cable being positioned in the elongated body;
a first member connected to the distal member, a portion of the first member being positioned in the elongated body and movable relative to the elongated body;
a stopper operable from one position permitting movement of the first member relative to the elongated body to an other position preventing movement of the first member relative to the elongated body;
a second member connected to the distal member, a portion of the second member being positioned in the elongated body and movable relative to the elongated body, the second member being either the cable or an elongated wire member; and
an operation lever connected to the second member so that the operation lever and the second member move together, the operation lever being movable to move the second member when the stopper is in the other position to change a direction toward which the medical-treatment member is directed.

2. The medical device according to claim 1, wherein the medical-treatment member is a camera, and the cable is a camera cable connected to the camera.

3. The medical device according to claim 1, wherein the first member is a light guide.

4. The medical device according to claim 1, wherein the light guide is positioned in a lumen in the distal member.

5. The medical device according to claim 1, wherein the stopper is a cap portion that is rotatable between the one position and the other position.

6. The medical device according to claim 1, wherein the stopper is a tongue portion that is movable in a proximal direction to sandwich the second member between the tongue portion and a center shaft portion in the elongated body.

7. A medical device comprising:
an elongated body possessing an open distal end;
a distal member provided with a medical-treatment member, the distal member being held by the elongated body in a freely movable manner in which the distal member moves both toward and away from the open distal end of the elongated body;
an operation member which includes a first member connected to the distal member to transmit a moving force to the distal member to move the distal member relative to the elongated body toward and away from the open distal end of the elongated body, the first member being configured to be curved so that the first member exhibits a curvature factor;
the operation member also including a second member connected to the distal member; and
wherein the curvature-factor by which the first member is curved is freely adjustable based on cooperation between the first member and the second member.

8. The medical device according to claim 7, wherein a direction toward which the distal end of the distal member is directed is freely adjustable through operation of the operation member.

9. The medical device according to claim 7, wherein the operation member adjusts the curvature-factor by which the first member is curved by moving the distal member relative to the elongated body and by adjusting an amount of protrusion by which the distal member protrudes distally beyond the opening portion.

10. The medical device according to claim 7, wherein the operation member: i) curves the first member by moving the distal member in the distal direction, by way of the first member, relative to the elongated body in a state in which a distance between the open distal end and the distal member is fixed by way of the second member; or ii) curves the first member by moving the second member in a proximal direction in a state in which the distance between the opening portion and the distal member is fixed by way of the first member.

11. The medical device according to claim 7, wherein
the first member and the second member are both wire-shaped bodies; and
the operation member further comprises a first stopper that fixes a position of the first member, and a second stopper that fixes a position of the second member.

12. The medical device according to claim 7, wherein:
the medical-treatment member of the distal member is a camera;
the wire-shaped body as the first member is constituted by a light guide which is attached to the distal member to illuminate while the camera is taking an image; and
the wire-shaped body as the second member is constituted by a camera cable connected to the camera.

13. The medical device according to claim 7, wherein
the first member is a flexible tubular body positioned in the elongated body and connected to the distal member;
the second member is constituted by a wire-shaped body; and
the operation member further comprises a stopper for fixing the elongated body with respect to the tubular body.

14. The medical device according to claim 13, wherein the distal member and the tubular body comprise a lumen for inserting another medical-treatment member.

15. The medical device according to claim 13, wherein
the tubular body possesses an outer circumferential surface and includes a recessed portion that is recessed relative to the outer circumferential surface of the tubular body to define a longitudinally extending groove in the elongated body; and
the wire-shaped body as the second member is housed in a space which is formed between the groove and the elongated body and is pulled out from inside of the groove toward the outside while also curving the first member connected to the distal member.

16. The medical device according to claim 13, wherein
the treatment member of the distal member is constituted by a camera, and
the wire-shaped body as the second member is constituted by a camera cable connected to the camera.

17. A method of changing a direction of orientation of a medical-treatment member comprising:
moving a distal member in a forward direction away from an open distal end of an elongated body, the medical-treatment member being mounted in the distal member so that the distal member and the medical-treatment member move together as a unit, a first member connected to the distal member to transmit a moving force to the distal member to move the distal member relative to the elongated body away from the open distal end of the elongated body, the first member being configured to be curved, and a second member connected to the distal member;
after moving the distal member in the forward direction away from the open distal end of an elongated body, fixing the first member relative to the elongated body so that movement of the distal member relative to the elongated body is prevented; and
moving the second member relative to the elongated body while the first member is fixed relative to the elongated body to curve the first member and change a direction toward which the medical-treatment member is directed.

18. The method according to claim 17, wherein the medical-treatment member is a camera which is connected to a camera cable, at least a portion of the camera cable being positioned in the elongated body.

19. The method according to claim 17, further comprising a light guide, a portion of the light guide being positioned in the elongated body, and a distal portion of the light guide being positioned in a lumen in the distal member.

20. The method according to claim 17, wherein the first member is fixed relative to the elongated body either by rotating a cap portion that surrounds a portion of the elongated body to inwardly press a portion of the elongated body against the first member or by proximally moving a tongue portion to sandwich the second member between the tongue portion and a center shaft portion in the elongated body.
